(19) United States
(12) Patent Application Publication
(10) Pub. No.: US 2022/0128721 A1
(43) Pub. Date: Apr. 28, 2022

(54) SENSOR CHIP FOR DETECTING LIGHT

(71) Applicant: Forschungszentrum Juelich GmbH, Juelich (DE)

(72) Inventors: Christoph Lerche, Herzogenrath (DE); Arne Berneking, Karlsruhe (DE); Nadim Joni Shah, Juelich (DE)

(21) Appl. No.: 17/422,761

(22) PCT Filed: Dec. 18, 2019
(86) PCT No.: PCT/DE2019/000331

§ 371 Date: Jul. 14, 2021

(30) Foreign Application Priority Data
Jan. 28, 2019 (DE) 10 2019 000 614.3

(51) Int. Cl. G01T 1/29; G01T 1/164

(52) U.S. Cl. CPC G01T 1/2985; A61B 6/037; G01T 1/1644

(57) ABSTRACT

A sensor chip includes a plurality of microcells to which an xy position is assigned, composed of a photodiode D_{n,m}, a current divider S_{q,nm}, with outputs S_{q,v,nm}, for the y direction and outputs S_{q,h,nm} for the x direction, the outputs S_{q,h,nm} being equipped with a quenching apparatus R_{q,h,nm} for quenching the current, and the outputs S_{q,v,nm} being equipped with a quenching apparatus R_{q,v,nm} for quenching the current, which divides the generated photocurrent of the diodes Dn,m into two equally large fractions. The microcells are arranged in a sequence of N columns in the x direction x_{n,}=x₁, x₂, x₃, . . . xₙ with n=1, 2, 3, . . . N and M rows in the y direction y_{m,}=y₁, y₂, y₃, . . . yₘ with m=1, 2, 3, . . . M. Outputs S_{q,h,nm} of the current dividers S_{q,nm} for the x direction are connected to the read-out channels Ch_{A} and Ch_{B} for the x direction.

### CROSS REFERENCE TO RELATED APPLICATIONS

This application is a U.S. National Phase application under 35 U.S.C. § 371 of International Application No. PCT/DE2019/000331, filed on Month Dec. 18, 2019, and claims benefit to German Patent Application No. DE 10 2019 000 614.3, filed on Jan. 28, 2019. The International Application was published in German on Aug. 6, 2020 as WO 2020/156600 A1 under PCT Article 21(2).

### FIELD

The present disclosure relates to a sensor chip suitable for a positron emission tomography detector ring.

### BACKGROUND

According to the prior art, positron emission tomography detector rings are used to detect β⁺β⁻ annihilation radiation. The rings consist of scintillation crystals adjoined by sensors that are capable of detecting scintillation radiation. Typical detectors are SiPMs (silicon photomultipliers). The structure is designed to be such that the detector ring is generally circular, wherein the object to be measured, for example a body part of a patient or an animal, is placed in the center of the detector ring (PET ring). Through the use of radiodiagnostics, β⁺β⁻ annihilation radiation is generated, which is to be detected. The β⁺β⁻ annihilation radiation strikes scintillation crystals arranged in a ring shape around the object to be examined and generates the scintillation radiation. The scintillation radiation, in turn, is registered by the SiMPs, which is located in the concentric arrangement behind the scintillation crystal relative to the radiation source. However, the SiMPs can also be arranged on other sides of the scintillation crystal, for example in front of the scintillation crystal or on the side thereof. The scintillation crystal is a three-dimensional body. Relative to an arrangement in which the object to be examined emits annihilation radiation from the center of the detector ring, the cross section on which the annihilation radiation strikes the scintillation crystal spans an xy plane. The depth of the scintillation crystal is referred to as a z axis in this nomenclature. In an idealized representation, at the center of the detector ring is an object to be examined or an emission source for radiation having an energy of 511 keV, which ideally strikes the xy plane of the scintillation crystal perpendicularly and has a penetration depth along the z axis of the scintillation crystal. The 511 keV annihilation radiation then triggers a scintillation at a point of the scintillation crystal along the z axis, which scintillation is registered by the sensor, for example a SiPM, as a signal. A SiPM is even capable of detecting individual photons. When the minimum required light strikes the active sensor surface, the SiPM microcell experiences a breakdown of the diode. The microcells are therefore also referred to as single-avalanche photodiode, SPAD. This generates a current pulse, which can be measured at the output of the component. A so-called quench resistor prevents the cell from generating a critical current which becomes so high that the component is destroyed. The output current of a SiPM microcell is independent of the amount of light that has reached the sensor and started the breakdown process. A SiPM microcell is a binary sensor that detects whether or not light is incident. In order to obtain quantitative information about the incident light, a SiPM consists of a plurality of microcells. A microcell is composed of a photodiode and a quench resistor. The number of broken-down cells then provides information about the amount of incident light.

There is a correlation between the sensitivity of the scintillation crystal and the length thereof along the z axis. The thicker (longer extension in the z direction) the scintillation crystal is dimensioned, the more sensitive it is since a scintillation event is all the more likely to occur.

During the detection of the annihilation radiation, beams are emitted in two opposite directions from the point at which the annihilation radiation is emitted, so that the beams form an angle of 180°. The line formed by these beams is referred to as the “line of response” (LOR). Accordingly, in the case of an annular detector, two beams strike scintillation crystals along the LOR, which scintillation crystals are situated on opposite sides relative to the annular arrangement at the center of which the emission source is located.

Various established methods for determining the x and y positions of an event exist for detectors having light detection by photodiodes in the form of SiPMs on only one side of the scintillation crystal. However, these methods do not include the z position, and thus the exact position in the scintillation crystal where the gamma photon was stopped on the z axis and converted to light (photoconversion) is not determined. If the z position is not determined as well, parallax errors occur during the determination of the LOR, which errors can be attributed to the described depth-of-interaction problem (DOI problem). The DOI problem arises whenever the LOR is not parallel to the z axis of the scintillator crystal. The further the emission center for an LOR is located outside the center of the transaxial plane of a PET ring, the greater the problem becomes. In the design of a PET ring, this results in a compromise between increasing the sensitivity due to longer scintillation crystals and reducing the DOI errors due to shorter scintillation crystals. In some areas of the PET application, there is a need to use PET rings (detector rings) resting closely against the examination object. This is the case in particular in medicine when patients are to be examined simultaneously by way of an MRI method and a PET method. The PET ring then has to fit into the opening of the MRI scanner tube. As a result, the PET ring that is used must be dimensioned to be small in diameter so as to fit into the opening of the MRI ring. In the case of a small dimensioning of the PET ring, however, there is the problem that, even though the object to be examined, for example a body part of a small animal or also of a human, can be arranged centered, it is dimensioned, relative to the diameter of the PET ring, so as to reach far into the edge regions of the opening of the PET ring. However, points from which annihilation radiation originates are thus also positioned so close to the PET ring that the DOI problem becomes significant.

In recent years, in particular the resolution in the case of small animal PET scanners has been significantly improved with the use of pixelated scintillation crystal blocks having ever smaller pixel sizes. In this case, the pixelation is implemented in the xy plane so that tubes of pixels oriented in the z direction form in the scintillation crystal. A reduction in the pixel size in the xy plane was especially promoted by the need for increasingly higher spatial resolution in small animal PET scanners since the object examined is very small. Meanwhile, the pixel size has already reached the submillimeter range. As a result, two problems increasingly occur which must be solved. First, the pixelated crystal blocks are composed of adhesive and reflector film which is located between the individual scintillation crystals in order to thus create a pixelated block having pixels that are optically isolated with respect to one another. The layer of adhesive and reflector film has an approximate thickness of 70 μm. Accordingly, pixelated arrays having a particularly low pixel pitch have an increased sensitivity loss. In the case of an array including crystal pixels measuring 0.8 cm×0.8 cm in size, as were used, for example, in [1], the ratio of the adhesive and film to the scintillation crystal is significantly reduced so that adhesive and film already account for a fraction of 29%. The scintillation crystal fraction is consequently reduced to 71%. In the other 29% volume, gamma quanta can be stopped and converted to light only very inefficiently. If even smaller pixelated arrays of, for example, 0.5 cm×0.5 cm are used, the crystal fraction is even reduced to 59%. The increase in resolution with pixelated arrays is therefore always tied to a loss of sensitivity. The second problem with pixelated scintillation crystal arrays is that the emitted light is concentrated on a smaller region of SiPM detector surface. A SiPM consists of multiple microcells which, as described above, function as binary elements. The more light strikes a SiPM, the higher is the probability that two or more light quanta strike the same microcell of the SiPM. These additional light quanta then cannot be detected. Consequently, the probability of saturation of a SIPM is significantly higher when pixelated scintillation crystal arrays are used, since they concentrate the light more strongly onto a small region of the SiPM. Saturation effects also lead to poorer energy resolution and temporal resolution of the detectors.

As mentioned at the outset, detectors of the prior art use SiPM-based sensor technologies to enable magnetic resonance imaging (MRI) compatibility for use in MR/PET hybrid scanners. Another problem with hybrid scanners is that the space for PET detectors and associated electronics is limited by the tube diameter of the magnetic resonance tomograph (MRT). This is especially true for ultra-high field tomographs. As a consequence of the narrower tube diameter, the PET scintillation crystals must be as short as possible. Shorter scintillation crystals also reduce sensitivity. This also means that the PET ring is located closer to the examination object due to the conditions of the tube diameter. Apart from limitations due to hybrid devices, attempts are also made to use PET rings having as small a diameter as possible due to higher sensitivity and lower cost.

Furthermore, it is known that many SiPM sensor concepts for PET devices include encoding of the output channels since increasing the output channels increases the power consumption of the PET ring. However, this increase is limited for design reasons. A simple calculation illustrates this. A PET ring having a diameter of 8 cm and a length of 10 cm results in a detector surface of 251 cm². If a 1-to-1 coupling of scintillation crystals and SiPMs with a crystal pixel size of 0.8 mm is used, 39270 read-out channels are already required if each channel is read out individually.

In order to achieve higher spatial resolutions, current sensor designs are made of sensor chips having smaller pixel sizes (i.e., made of multiple, independent SiPMs arranged in a matrix). In this case, a pixel of the sensor chip denotes multiple individual microcells connected in parallel. This leads to a significant increase in the read-out channels, which are limited by the power consumption, space, and data rates. As a consequence, position-sensitive (PS) encoding methods were developed in order to reduce the number of read-out channels of a chip [1-3, 15]. The most recently developed concept is PS-SSPM [1] and is based on charge-sharing PS-SiPMs. Charge-sharing PS-SiPM microcells detect the light similarly to conventional SiPM microcells. However, this sensor concept includes a resistor network which distributes the generated charge as a function of the position and the encoding. The detector structure presented in [1] consists of a pixelated crystal array having a distance of 0.8 mm.

This most recent detector concept enables the advantage of an output channel reduction by channel encoding, with a high detector array resolution at the same time, which is achieved through the use of pixelated scintillation crystal arrays having a distance of less than one millimeter. However, it does not include DOI information detection.

A concept published in [4] proves the possibility of creating a PET detector consisting of monolithic crystals and SiPMs. As already mentioned above, monolithic crystals solve the problem of sensitivity losses due to the space requirement of reflector films and associated adhesives. Moreover, the production costs of monolithic crystals are lower due to the elimination of cutting to size and bonding of the scintillator pixels. The thickness of the crystals used is 2 mm. As a result, parallax errors are avoided by the structure used in [4], which however comes with the disadvantage of the small extension of the scintillation crystal in the z direction. At the same time, however, the detection efficiency is low due to the low crystal height.

There are various options for measuring DOI information and thus correcting parallax errors, which additionally detect light at a further crystal side. Particularly for SiPMs of the prior art, costs thus increase greatly. A concept for DOI detection, which only detects light on one crystal side and uses monolithic crystals in the process, is published in [5] and patented in [6]. This uses the known principle that the light distribution of the crystal is a function of the DOI. The detector concept used is coupled with monolithic crystals on position-sensitive photomultipliers (PMT) H8500 by Hamamatsu. In addition, a resistor network is used, which enables position encoding, and thus also output channel reduction. The standard deviation of the light distribution is used to estimate the DOI. The 1st and 2nd order moments of the light distribution are required to calculate the standard deviation. The 1st order moment is already given by the linear encoding of the output channels. In order to determine the 2nd order moment, a sum network has been developed and integrated into the resistor network.

An overview of PET detectors including DOI detection is summarized in [7]. Descriptions and results of small animal PET and MR/PET hybrid scanners developed in recent years can be found in [8-11].

Detector concepts which are based on current SiPM-based technology and include position encoding for channel reduction do not include DOI detection. For this reason, PET rings made with these detectors contain parallax errors in the reconstructed images. Moreover, most scintillation detectors use pixelated scintillator crystal arrays. As described above, this leads to a loss of sensitivity due to the reflector film and the adhesive between the crystals of the array. Due to the lack of DOI information, there are limitations when it comes to the thickness of the crystals. An increase in the sensitivity due to thicker crystals is accompanied by a loss in spatial resolution due to the lack of DOI information and the resulting parallax errors. The DOI concepts for pixelated crystals mentioned in [7] can, in principle, also be implemented with arbitrarily small scintillator crystals; however, the described disadvantages, such as saturation effects and sensitivity loss, also apply to these concepts.

Currently, SiPM sensors are one of the most expensive components of a PET ring.

The concept implemented in [5, 6] uses position-sensitive PMT, which cannot be used in strong magnetic fields. As a result, they are not MRI-compatible. The concept could be implemented with MRI-compatible avalanche photodiodes (APD), which has not yet happened to date. APDs are photodiodes operated by applying a suitable bias voltage in the proportional operating range. A charge carrier pair generated by an optical photon generates further charge carrier pairs (charge carrier avalanches) through repeated secondary ionization. The resulting photocurrent depends on the light intensity, as is the case with PMTs. Nevertheless, an implementation of this concept at the SiPM microcell level is another challenge since SiPM microcells are binary sensors and are operated in another mode, the so-called Geiger mode.

The possibility of DOI detection using position-sensitive PMTs has been proven in [10, 11].

Research results using detectors consisting of SiPMs and monolithic crystals are published in [12]. In this approach, SiPMs are used in the same way as the original concept published for PMTs and APDs in [5, 6].

German patent applications 10 2016 006 056, 10 2016 014 113 and 10 2016 008 904 disclose sensor chips with which the DOI problem can be solved or reduced.

In the sensor chip disclosed in German patent application 10 2016 006 056, the encoding resistors and the resistors used for the current divider have to be significantly smaller, i.e., at least by a factor of 100, better 1000, than the summing resistors, which in turn have to be significantly smaller than the quench resistors, i.e., by a factor of 100, better 1000. As a result, the number of microcell positions to be encoded is limited due to the limited available space on the sensor chip. However, the sensor chip disclosed there is based on encoding as many individual microcells as possible. In addition, this should be ensured for the largest possible photosensitive sensor surface (i.e., large extension in the x and/or y directions). Furthermore, two encoding resistors are required for each x and/or y direction. Moreover, the more microcell positions along the x or y direction have to be encoded, the increasingly smaller the difference or the discrepancy in size in relation to the neighboring resistors becomes. This leads to limitations of the encoding method carried out with the sensor chips. This method can likewise be limited by technical production limitation, where it is no longer possible to precisely integrate resistor sizes or it is more complicated to implement resistances of many different values. The quench and summing resistors each have the same values, in contrast to the encoding resistors, which is easier to implement with common integrated circuit (IC) production technologies.

A further disadvantage is that produced ICs having the same encoding of encoding resistors cannot be combined with one another and their channel without irretrievably deactivating the position encoding and the depth-of-interaction encoding. Interconnecting multiple sensor chips with a small sensor surface to form a larger unit with a large sensor surface while maintaining the correct position encoding and the depth-of-interaction encoding is very advantageous since the production yield per unit area is greater for sensor chips with a small surface than for sensor chips with a large surface. This has a very advantageous effect on the production unit costs. Furthermore, the resistors take up a relatively large amount of space on the IC, which is why the available space for photodiodes is reduced, which leads to a reduction of the photosensitive surface, and thus of the photodetection efficiency (PDE).

### SUMMARY

In an embodiment, the present disclosure provides a sensor chip. The sensor chip includes a plurality of microcells to which an xy position is assigned, composed of a photodiode D_{n,m}, a current divider S_{q,nm}, with outputs S_{q,v,nm}, for the y direction and outputs S_{q,h,nm} for the x direction, the outputs S_{q,h,nm} being equipped with a quenching apparatus R_{q,h,nm} for quenching the current, and the outputs S_{q,v,nm} being equipped with a quenching apparatus R_{q,v,nm} for quenching the current, which divides the generated photocurrent of the diodes Dn,m into two equally large fractions. The microcells are arranged in a sequence of N columns in the x direction x_{n,}=x₁, x₂, x₃, . . . xₙ with n=1, 2, 3, . . . N and M rows in the y direction y_{m,}=y₁, y₂, y₃, . . . yₘ with m=1, 2, 3, . . . M. The outputs S_{q,h,nm} of the current dividers S_{q,nm} for the x direction are connected to the read-out channels Ch_{A} and Ch_{B} for the x direction, current conductors of the same x position of the sensor chip being connected to the same signal bus N_{S,h,i}, which leads into the read-out channel Ch_{A} and Ch_{B} in the x direction. A series connection of x-encoding resistors R_{h,0}, Rₕ, R_{h,2}, . . . . . R_{h,N} is located in the read-out channels Ch_{A} and Ch_{B}, the signal buses N_{S,h,i} leading into nodal points K_{h,n} with n=1, 2, 3, . . . N, which are located between the x-encoding resistors R_{h,0}, R_{h,1}, R_{h,2}, . . . R_{h,N}, thereby effecting linear encoding, the linear encoding being given when the following condition is satisfied:

Q₁(ε)=c₁·ε^{c2}+c₃

Q₂(ε)=c₄·ε^{c3}+c₆

c₁=const.∈(0,∞)

c₄=const.∈(−∞,0)

c₃,c₆=const. ∈(−∞,∞)

0.5<c₂,c₅<1.5 (Formula 1)

### BRIEF DESCRIPTION OF THE DRAWINGS

Subject matter of the present disclosure will be described in even greater detail below based on the exemplary figures. All features described and/or illustrated herein can be used alone or combined in different combinations. The features and advantages of various embodiments will become apparent by reading the following detailed description with reference to the attached drawings, which illustrate the following:

FIG. 1 provides a representation in which individual microcells are connected via signal buses to the output channels with the linear encoding;

FIG. 2 illustrates an embodiment in which four SPADs are combined with associated quench resistors to form a microcell;

FIG. 3 illustrates a summing circuit consisting of summing networks and downstream summing amplifiers;

FIG. 4 illustrates an embodiment in which 4 sensor chips are connected over an entire row and column;

FIG. 5 illustrates a representation as in FIG. 1 with summing networks;

FIG. 6 illustrates a summing amplifier as an external circuit for summing networks integrated on the sensor chip; and

FIG. 7 illustrates a representation as in FIG. 4 with summing networks implemented on the sensor chip.

### DETAILED DESCRIPTION

The present disclosure provides a sensor chip which overcomes the disadvantages of the prior art, with which the parallax errors during the determination of an LOR can be reduced. A sensor chip is to be provided which enables the use of scintillation single crystals for the detection of signals during positron emission tomography, wherein the DOI problem can be avoided by reducing the parallax error during the determination of the LOR.

The sensitivity and the resolution of the sensor chip are to be improved. Furthermore, the sensor chip is to be suitable to be operated together with an MRT, in particular with high magnetic fields and small magnet inside diameters. The accuracy of PET rings that are small-dimensioned, or in the case of PET rings that rest closely against the examination object, is to be improved. The space required by the electronics associated with the measuring system is to be reduced. The costs for the device are to be reduced. In its application, the sensor chip is not to be limited to the use in PET but should generally be usable for scintillation single crystals. Furthermore, the number of microcell positions to be encoded is to be increased. Encoding is to be made possible over a larger number of microcells than according to the prior art, wherein the limitation by resistance values achievable on the IC is to be reduced or eliminated. The area required by the resistors is to be reduced so that more space is available on the chip for microcells or SPADs. The present disclosure provides for simultaneously achieving both linear encoding of the microcell currents and quadratic encoding of the voltage drops generated by the microcell currents according to their xy position, wherein the encoding is to be achieved in particular across the boundaries of an individual sensor chip in order to achieve a successful determination of the position and depth of the detection position in the monolithic crystal of the scintillation detector, when monolithic crystals are used that are larger than a single sensor chip, and for this reason multiple sensor chips are required for registering the scintillation light. The sensor chip is to enable light detection, in particular in the IR, in the visual and UV range.

With the sensor chip, it is now possible to reduce parallax errors during the determination of the LORs, in particular in the case of scintillation single crystals. The sensitivity and the resolution of the measuring method and of the device are improved. The use of scintillation single crystals that are longer in the z direction is to be made possible.

The detector can be operated together with an MRT device. The parallax error is reduced, in particular in the case of devices with small dimensioning or when the PET ring rests closely against the examination object. Space for the associated electronics and costs are saved. The sensor chip according to the invention achieves a very high level of detail accuracy. The reason is that the number of scans of the light distribution function is significantly increased as a result since even scanning at the microcell level is possible. This increases the granularity that is available for determining the 2nd moment by a factor that, depending on the implementation methods described later, can be up to 160 or higher compared to conventional SiPMs, photomultipliers or avalanche diodes. This leads to a more precise determination of the 2nd order moment. Furthermore, the number of microcell positions to be encoded is increased. Encoding is made possible over a larger area of microcells than according to the prior art, wherein no limitation of resistor sizes exists or the limitation is reduced. The space that the resistors take up on the chip is reduced, which is why the available space for photodiodes is increased. Sensor chips with larger photosensitive surfaces (larger number of microcells) can be encoded. Linear encoding of the currents and quadratic encoding of the voltage drops are possible across multiple sensor chips. Light detection in the IR, visual and UV range is made possible.

According to the disclosure, linear encoding of currents is carried out in the case of a sensor chip in one read-out direction x or in two read-out directions x and y, wherein the linear encoding in the x and/or y directions takes place by a series connection of encoding resistors.

In this case, the linearly encoded signal for the x direction can be tapped at the outputs Ch_{A} and Ch_{B}, and for the y direction at the outputs Ch_{C} and Ch_{D}. This results in approximately linear, increasing or decreasing dependencies between the signals at the output and the xy position at which a signal injection by the microcells into the encoding network takes place.

The method can be carried out with all photosensors that contain location encoding, wherein the encoding should correspond to linear encoding to the extent possible.

In this case, the output signal of one channel or a combination of channels must change to ascend as linearly as possible with the x or y position, while the output signal of another channel or a combination of channels changes to descend as linearly as possible with the x or y position. Linear encoding is to be understood to mean any encoding that corresponds to Formula 1. Here, Q1 is the charge of the output channels ascending over the e position, and Q2 is the charge of the output channels descending over the e position. The variable e denotes the encoding direction, i.e., x or y; c₁₋₆ denote constants.

Q₁(ε)=c₁·ε^{c2}+c₃

Q₂(ε)=c₄·ε^{c3}+c₆

c₁=const.∈(0,∞)

c₄=const.∈(−∞,0)

c₃,c₆=const. ∈(−∞,∞)

0.5<c₂,c₅<1.5 (Formula 1)

The parentheses for the expressions c₁, c₄, c₃ and c₆ in Formula 1 are open intervals in the mathematical sense.

Formula 1 takes into consideration that embodiments which do not satisfy the requirements with regard to exact linearity, i.e., produce only approximately linear encodings, may still be suitable for implementing the teachings described herein. Ideally, the linear encoding is exactly linear. In the case of exact linearity, c₂=1 and c₅=1.

Depending on the position of the microcell, the photocurrent is divided among the outputs and flows into positions within the series connections which have a number of encoding resistors Rₕ corresponding to the position to be encoded in directions x, or Rᵥ in the y direction. Depending on this position, the photocurrents are distributed among the outputs Ch_{A}, Ch_{B}, Ch_{C} and Ch_{D} since, depending on the position, more or fewer resistors are located between the microcell position and the corresponding outputs, and the total resistance to the corresponding outputs thus varies with the position. In this case, only N+1 equally large resistors are required for N× positions, or only M+1 equally large resistors are required for M y positions.

In the following, terms relating to resistance are defined.

Resistance value is to be understood to mean the nominal value of the resistance in ohm. In the case of resistance materials with equal electrical conductivity, the resistance values are equally large at the same resistance geometry. In the case of resistance values with different electrical conductivities, the resistance values are differently large at the same resistance geometry. In the case of resistance materials with the same conductivity and different resistance geometries, the resistance values are also differently large.

The term “resistance” denotes the physical resistance as a bodily object having a functional designation, without the resistance value being nominally established thereby.

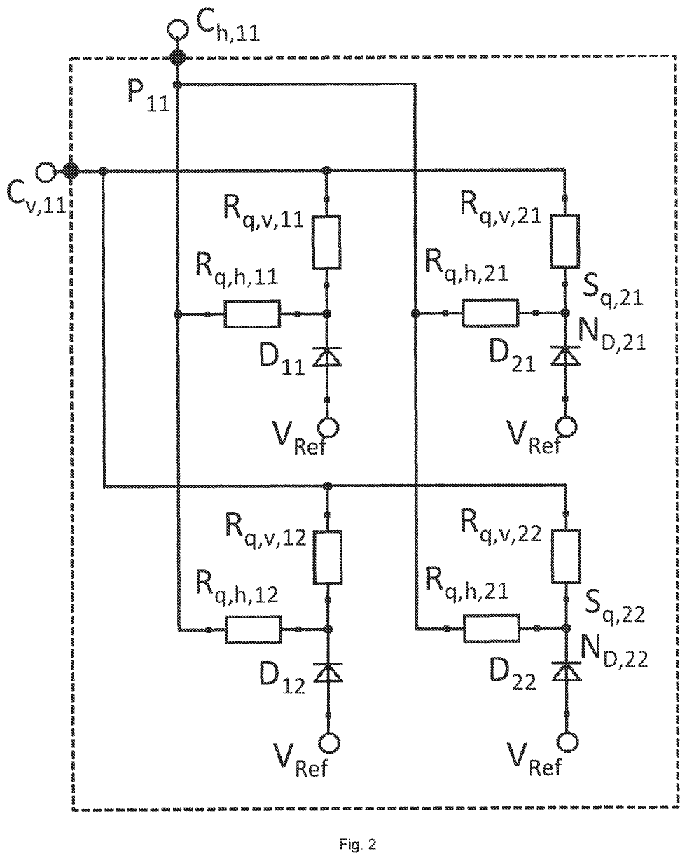

The sensor chip comprises a plurality of microcells, which are characterized in that a dedicated (x,y) position is assigned to each microcell. A microcell consists of at least one photodiode D_{n,m} and a current divider S_{q,nm}, with outputs S_{q,v,nm}, for the y direction and outputs S_{q,h,nm} for the x direction, with means for quenching, for example quench resistors R_{q,h,nm} and R_{q,v,nm}, which divides the generated photocurrent of the diodes into two equally large fractions. Alternatively, multiple photodiodes D_{n,m} . . . D_{n+1,m+k} can be combined with associated current dividers and quench resistors to form a microcell, where I and k are arbitrary numbers as needed. (FIG. 2)

In the abbreviations mentioned for the current dividers S and the quench resistors R_{q}, the subscript h means that the corresponding signal buses lead to the output Ch_{A}, Ch_{B}, for the identification of the x position, and the subscript v means that the corresponding signal buses lead to the output Ch_{C}, Ch_{D} for the identification of the y position.

In particular single avalanche photodiodes (SPADs) can be used as photodiodes, wherein the quench resistors simultaneously assume the function of the current divider.

Instead of a quench resistor, the quenching process can also be initiated by active quenching, using the methods known to the person skilled in the art, or means for quenching, for example using a transistor and a comparator. In the following description, a quench resistor Rq or a current divider S implemented with the quench resistors R_{q,v,nm} and R_{q,h,nm} is disclosed in the disclosed embodiments. However, a different equivalent means for quenching, for example a transistor or comparator, can also be used in all embodiments so that the disclosure is not limited to the use of a quench resistor.

The microcells are arranged in a grid in which the microcells are arranged in rows in the x direction and in the y direction. The microcells are preferably arranged in rows or columns parallel to the x axis and the y axis. Typically, 10, 50, 100 or 1000 microcells are arranged in the x direction and the y direction, respectively. The arrangement then includes N columns in the x direction xₙ=x₁, x₂, x₃, . . . x_{N}, with n=1, 2, . . . N and M rows in the y direction yₘ=y₁, y₂, y₃ . . . y_{M}, with m=1, 2, . . . . M. Moreover, N and M are in each case also the number of microcells in the x and y directions. The directions x and y are preferably arranged orthogonally to each other, but they can also be arranged at an angle that deviates from 90° so that a rhombic pattern is produced.

This arrangement forms a block. A sensor chip can comprise a plurality of blocks, which are arranged in a grid. A block can be accommodated on the same substrate (or wafer, or chip), or on different ones.

The outputs of the current dividers S_{q,nm} implemented with the quench resistors are connected via the connections C_{h,nm} and C_{v,nm} to signal buses N_{S,h,n} for the x direction, and signal buses N_{s,v,m} for the y direction. The signal buses N_{S,h,n} lead into the nodal points K_{h,n} (n=1, 2, 3 . . . N) and, via a series connection of encoding resistors R_{h,0}, R_{h,1}, . . . R_{h,N}, are connected to the outputs Ch_{A}, Ch_{B}. The signal buses N_{S,v,m} join at the nodal points K_{v,m} (m=1, 2, 3 . . . . . M) and, via a series connection of encoding resistors R_{v,0}, R_{v,1}, . . . R_{v,NM}, are connected to the outputs Ch_{C}, Ch_{D}.

In addition, an electrical connection of the signal buses N_{s,v,0}, . . . N_{s,v,M} and N_{s,h,0} . . . N_{s,h,N} between different sensor chips and with external electronic or electrical circuits can be made possible via contacts of the sensor chip.

The first outputs of all current dividers in the same column n of the sensor chip are connected to the same signal bus N_{S,h,n} of the sensor chip. Thus, all signals from one column of the sensor chip reach the same signal bus, which in addition leads at the nodal point K_{h,n} into the series connection of encoding resistors R_{h,0}, R_{h,1}, . . . R_{h,N}.

The second outputs of all current dividers in the same row h of the sensor chip are connected to the same signal bus N_{S,v,m} of the sensor chip. Thus, all signals from one column of the sensor chip reach the same signal bus, which in addition leads at the nodal point K_{v,m} into the series connection of encoding resistors R_{h,0}, R_{h,1}, . . . R_{v,M}.

With the series connection of the encoding resistors R_{h,0}, R_{h,1}, . . . R_{h,N}, the linear encoding of the currents in the horizontal read-out direction (i.e., the x direction) is ensured. With the series connection of the encoding resistors R_{v,0}, R_{v,1}, . . . R_{v,M}, the linear encoding of the currents in the vertical read-out direction (i.e., the y direction) is ensured.

In order to effect linear encoding of the currents in the x direction, the encoding resistance values R_{h,1}, . . . R_{h,N-1} must have the same value Rₕ. In order to effect linear encoding of the currents in the y direction, the encoding resistance values R_{v,1}, . . . R_{v,M-1} must have the same value Rᵥ.

If the numbers of pixels in the x direction and y direction are different, the encoding resistance values can differ for the x direction and the y direction.

The number of encoding resistors N for Rₕ and M for Rᵥ per sensor chip is at least two and can take on values of 0.001 ohm to 100 Mohm. The number is in this case rather limited by practical circumstances.

The encoding resistance values for encoding Ch_{A} and Ch_{B} of the x direction and Ch_{C} and Ch_{D} corresponding to the y direction can be differently large. This can be advantageous, for example, if different numbers of microcells are present in the x and y directions so that the sensor chip or the microcells deviate from the square shape. In this case, the encoding resistance values encoding the larger number of pixels can be smaller than those along the other direction in which not as many pixel positions are to be encoded. In one embodiment, the sums of the encoding resistance values can be the same for the two directions x and y.

In order to enable multiple sensor chips to be connected among one another, the resistance values R_{h,0} and R_{h,N} must have the same value Rₕ/2. In order to enable multiple sensor chips to be connected among one another, the resistance values R_{v,0} and R_{v,M} must have the same value Rᵥ/2. Once the connection is established, the two resistances Rₕ/2 or Rᵥ/2 add up to form a resistance Rₕ or Rᵥ.

Overall, only N+1 or M+1 encoding resistors are required for N or M x or y positions.

The X and Y mean value of the light distribution detected by the active sensor surface of the sensor chip can be calculated according to

<X>=(B−A)/(A+B)

<Y>=(C−D)/(C+D).

The total amount of light E is determined as

<E>=A+B+C+D.

A, B, C, D are the signals that can be tapped via the outputs Ch_{A}, Ch_{B}, Ch_{C}, and Ch_{D}. They are generally currents; they may be charges if the currents are integrated by corresponding electronic components over time intervals.

When using the sensor chip with a scintillator, <E> is proportional to the energy of the detected gamma photon. After calibration, <X> and <Y> provide the x and y positions of the photoconversion within the active sensor surface of the sensor chip.

In order to increase the active sensor surface of the sensor chip, the sensor chip can be increased.

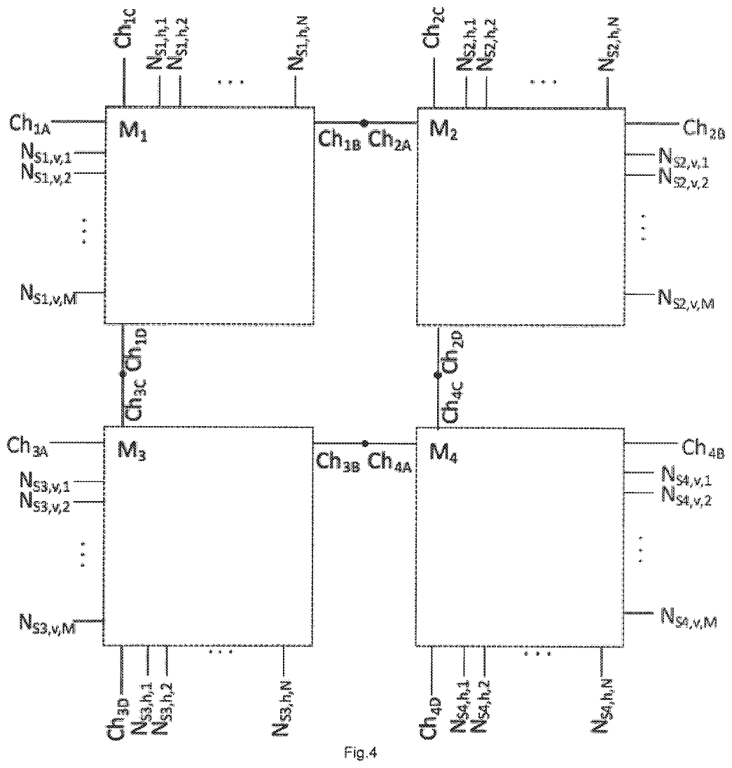

Alternatively, identical chips produced in the same way can be combined without any problems. In this case, the output channels Ch_{A}, Ch_{B}, Ch_{C} and Ch_{D} of the various sensor chips are connected to one another in such a way that in each case only two channels have to be read out at the two ends of the chip per encoding direction. (FIG. 4) This makes it possible to encode over even larger surfaces since the surface to be encoded is no longer limited by the encoding resistor sizes.

For each pixel column x and/or pixel row y of the two embodiments, the potentials are tapped at the signal buses N_{S,h,1}, N_{S,h,2} . . . N_{S,h,N} or N_{S,v,1}, N_{S,v,2} . . . N_{S,v,M} via the summing resistors R_{S,h,n} or R_{S,v,m} and fed into a summing network N_{S,h} or N_{S,v} including a downstream summing amplifier Oₕ or Oᵥ with the output channels Ch_{E} and Ch_{F}. An embodiment is possible in which the signal buses N_{S,h,1}, N_{S,h,2}, . . . N_{S,h,N} for the x direction and/or the signal buses N_{S,v,1}, N_{S,v,2}, . . . N_{S,v,M} for the y direction are connected to an external summing circuit consisting of summing networks N_{S,h} and N_{S,v}, downstream summing amplifiers Oₕ and Oᵥ with the output channels Ch_{E} and Ch_{F}. The resistance values for the summing resistors R_{S,h,n} or R_{S,v,m} are in each case equally large in a summing network N_{S,h} or N_{S,v}. The summing resistance values can be between 1Ω and 100 MΩ. The summing resistors R_{S,h,n} or R_{S,v,m} must be large enough that the generated photocurrent is not substantially influenced by the microcells but small enough not to influence the quench behavior of the microcells. The summing resistors R_{S,h,n} or R_{S,v,m} are combined via the signal buses of the summing networks N_{S,h} or N_{S,v}. The signals are thus summed up. The summing amplifiers Oₕ and/or Oᵥ can include an operational amplifier OPₕ or OPᵥ, which is grounded and has negative feedback with a resistor R_{S,h} or R_{S,v}. The amplification of the signal of the output channels Ch_{E} and Ch_{F} can be set via the ratio of R_{S,h}/R_{S,h,n} or R_{S,v}/R_{S,v,m}. The summing circuit consisting of summing networks N_{S,h} and N_{S,v} of downstream summing amplifiers Oₕ and Oᵥ can be integrated into the sensor chip, or parts thereof can in each case be, less preferably, situated outside of the sensor chip. In particular, the resistors R_{S,h,1}, . . . , R_{S,h,n} and R_{S,v,1}, . . . , R_{S,v,m} can be integrated onto the sensor chip so that only the summing networks N_{S,h} and N_{S,v} must be led out of the relevant sensor chip as signal buses and can be connected to external summing amplifiers Oₕ and/or Oᵥ. If the entire summing circuit consisting of summing networks N_{S,h} and N_{S,v} of downstream summing amplifiers Oₕ and Oᵥ is located outside the sensor chip, this results in all networks N_{S,h,n} and/or N_{S,v,m} being led out of the sensor chip as signal buses, which results in a very high number of output channels. If the summing resistors R_{S,h,n} and/or R_{S,v,m} are integrated into the sensor chip, only the summing networks N_{S,h} and/or N_{S,v} need to be led out of the sensor chip, which can be implemented with one output channel per network in each case. In order to enable the interconnection of multiple sensor chips, it is therefore preferable not to integrate the complete summing circuit consisting of summing networks N_{S,h} and N_{S,v} of downstream summing amplifiers Oₕ and Oᵥ into the sensor chip and to lead the signal buses N_{S,h,1}, N_{S,h,2}, . . . , N_{S,h,N} and N_{S,v,1}, N_{S,v,2}, . . . , N_{S,v,M} out of the sensor chip but to only integrate the resistors R_{S,h,1}, . . . , R_{S,h,n} and R_{S,v,1}, . . . , R_{S,v,m} onto the sensor chip and to only lead the summing networks N_{S,h} and N_{S,v} out of the relevant sensor chip.

The potentials Φ(N_{S,h,n}) or Φ(N_{S,v,m}) at the signal buses N_{S,h,n} and N_{S,v,m} should each have, as precisely as possible, a quadratic dependence of the position of the photocurrents of the microcells in the x and y directions. This is required in order to obtain the 2nd order moment of the signal distribution along the x direction and along the y direction. This is approximately ensured when the resistance values of the summing resistors R_{S,h,1} . . . , R_{S,h,n} and R_{S,v,1}, . . . , R_{S,v,m} are significantly greater (>factor 10) than the resistance values of the resistors R_{h,0}, R_{h,1}, . . . R_{h,N} and R_{v,0}, R_{v,1}, . . . R_{v,M}. In this case, the current flowing across the resistors R_{S,h,1}, . . . , R_{S,h,N} and R_{S,v,1}, . . . , R_{S,v,M} is negligible compared to the current flowing across the series connections R_{h,0}, R_{h,1}, . . . , R_{h,N} and R_{v,0}, R_{v,1}, . . . R_{v,M}. The outputs Ch_{A}, Ch_{B}, Ch_{C}, and Ch_{D} are preferably connected to the inputs of external amplifiers (not integrated on the chip) with very low input impedance, which is why the potential of the outputs Ch_{A}, Ch_{B}, Ch_{C}, and Ch_{D} relative to the nodal points K_{h,1}, . . . , K_{h,N} and K_{v,1}, . . . , K_{v,M} is 0, i.e., connected to ground. The total resistance which the current on the signal bus N_{S,h,i} sees at the i-th position along the x direction is then:

$${R}_{{K}_{h , i}} = \frac{( {\sum }_{l = 0}^{l = i} {R}_{h , l} ) ( {\sum }_{l = i + 1}^{l = N} {R}_{h , l} )}{{\sum }_{l = 0}^{l = N} {R}_{h , l}} = \frac{( i + \frac{1}{2} ) ( N - i + \frac{1}{2} ) {R}_{h}^{2}}{N \cdot {R}_{h}}$$

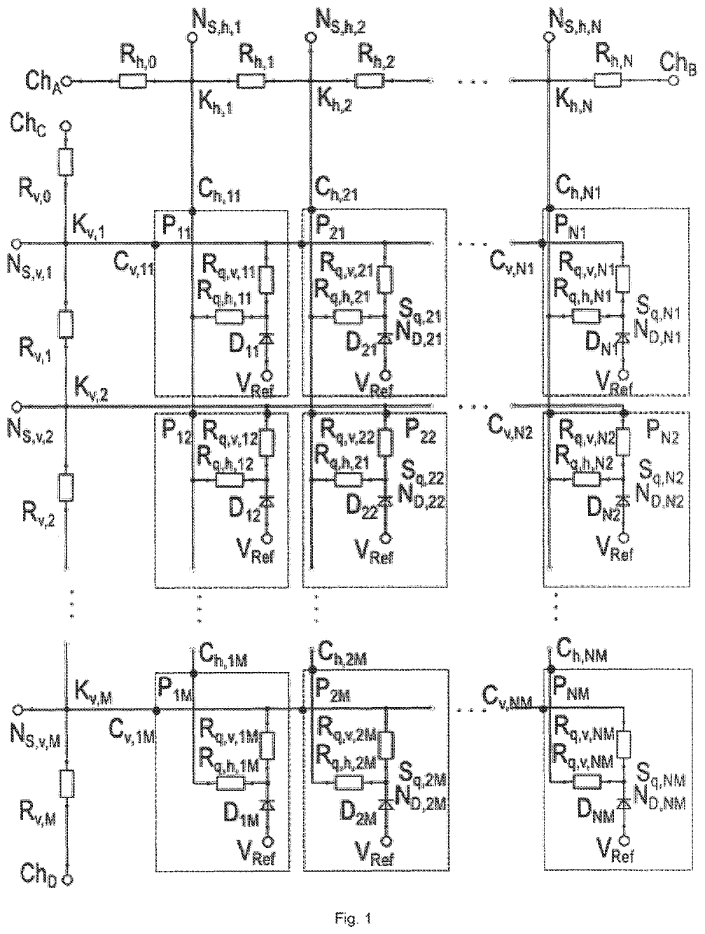

It depends quadratically on the position i. Due to Ohm's law U=R*I, the potentials at the nodal points K_{h,i} which result from the product of R_{K}_{_{h,i}} and the current coming from the microcells connected to the signal bus N_{S,h,i} are then also quadratically encoded in the position. As described in [5], the series connection R_{h,0}, R_{h,1}, . . . R_{h,N} forms a voltage divider for the current injected into the nodal points K_{h,i}, which leads to additional added voltage contributions, which ultimately, however, only as a proportionality factor N/2 independent of i. Equivalent observations apply to N_{S,v,1} at the i-th position along the y direction. When using the resistance values R_{h,0}=R_{h,N}=Rₕ/2 and R_{v,0}=R_{v,M}=Rᵥ/2 and R_{h,i}, . . . , R_{h,N-1}=Rₕ and R_{v,i}, . . . , R_{v,M-1}=Rᵥ, a total quadratic resistance value arises automatically at the nodal points K_{h,1}, . . . , K_{h,N} and K_{v,1}, . . . , K_{v,M} and thus the required quadratic potential distribution in the signal buses N_{S,v,m} and/or N_{S,h,n}. The resulting signals at the outputs Ch_{E}, Ch_{F} of the summing networks Oₕ and Oᵥ are proportional to the width of the light distribution striking the sensor chip. The width of the light distribution strongly correlates to the depth of interaction of the gamma photon and therefore allows the determination of the circuits in FIGS. 1-7 after calibration thereof. At the same time, the linear encoding for the photocurrent is given, which allows a determination of the interaction position in the xy plane via the outputs Ch_{A}, Ch_{B}, Ch_{C} and Ch_{D}. The potentials Φ(N_{S,h,n}) or Φ(N_{S,v,m}) at the signal buses N_{S,h,n} and N_{S,v,m} can also deviate from an exact quadratic encoding due to corresponding additional resistances or due to modified encoding resistors. Here, (Φ²ⁿ)^{k} with n=1, 2, 3 . . . and 0.5<k<1.5 must apply to the resulting potential encoding.

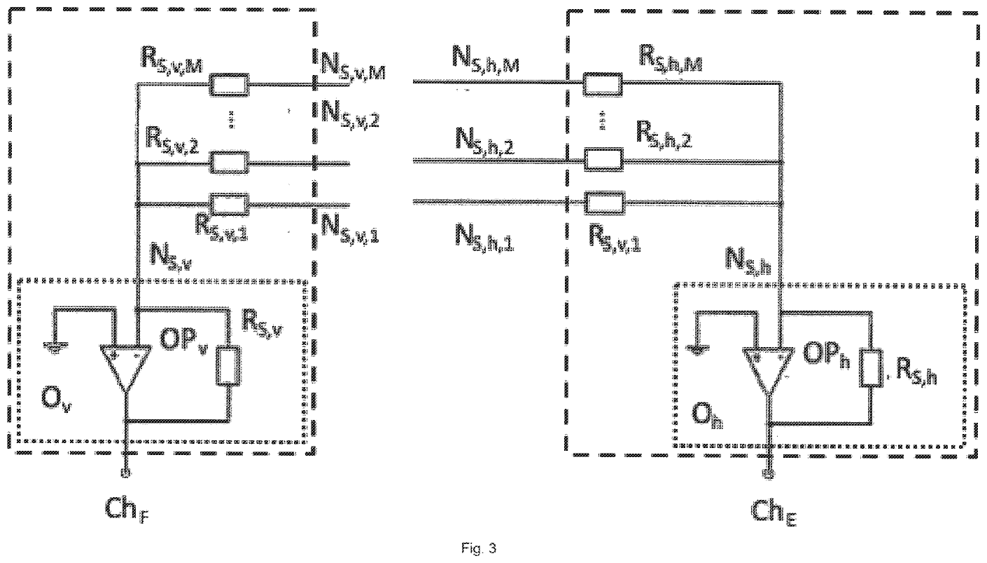

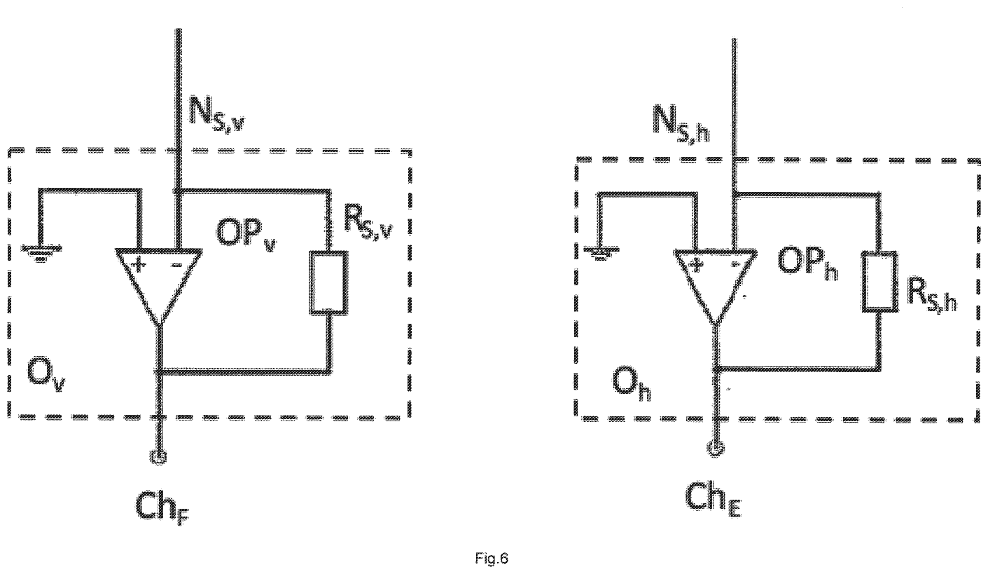

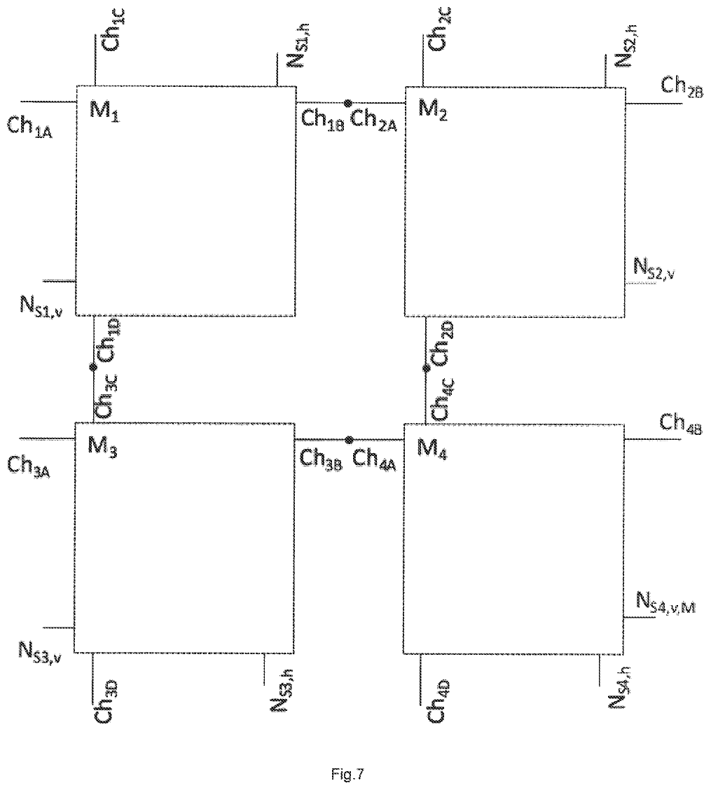

The implementation of the described linear and quadratic encoding with the aid of the series connection R_{h,0}, R_{h,1}, . . . R_{h,N} and R_{v,0}, R_{v,1}, . . . R_{v,M}, of the lead-out of the signal buses N_{S,h,1}, N_{S,h,2} . . . N_{S,h,N} and N_{S,v,1}, N_{S,v,2} . . . N_{S,v,M} or of the integration of the summing resistors R_{S,h,1}, . . . , R_{S,h,N} and R_{S,v,1}, . . . , R_{S,v,M} on the sensor chip and of the lead-out of the signal buses N_{S,h} and N_{S,v}, and the use of external summing networks Oₕ and Oᵥ or external operational amplifiers with feedback resistors R_{S,h} and R_{S,v} (FIG. 6) allow multiple sensor chips according to FIGS. 3 and 7 to be interconnected while maintaining the information about the depth of interaction and the interaction position along the xy plane.

The potentials Φ(N_{S,h,n}) or Φ(N_{S,v,m}) at the networks N_{S,h,n} and N_{S,v,m} can also deviate from an exact quadratic encoding due to corresponding additional resistors or due to modified encoding resistors. Here, (Φ²ⁿ)^{k} with n=1, 2, 3 . . . and 0.5<k<1.5 must apply to the resulting potential encoding.

FIG. 1 shows microcells comprising photodiodes Dₙₘ, which lead into a current divider S_{q,nm}, which is implemented with the quench resistors R_{q,h,nm}, R_{q,v,nm}. The outputs of the current dividers R_{q,h,nm} lead into signal buses N_{S,h,n}, which lead into the nodal points K_{h,n} and, via the series connection R_{h,0}, R_{h,1}, . . . R_{h,N}, into the output channels Ch_{A} and Ch_{B}. The outputs of the current dividers R_{q,v,nm} lead into signal buses N_{S,v,m}, which lead into the nodal points K_{v,m} and, via the series connection R_{v,0}, R_{v,1}, . . . R_{v,M}, into the output channels Ch_{C} and Ch_{D}. A series connection including the resistors R_{h,0}-R_{h,N} R_{v,0}-R_{v,M} is present in the outputs Ch_{A} and Ch_{B} or Ch_{C} and Ch_{D}.

In FIG. 2, four SPADs with associated quench resistors are assembled to form a microcell. In this figure, the same elements of the microcell have the same designations as in FIG. 1. Within this sensor, all outputs of the current dividers with the encoding resistors R_{h,nm} lead into a point C_{h,11} which is connected to the input of a signal bus N_{S,h,n}, which is not shown. Within this sensor, all outputs of the current dividers with the resistors R_{v,nm} lead into a point C_{v,11} which is connected to the input of a signal bus N_{S,v,M}, which is not shown.

FIG. 3 shows summing circuits in which the summing networks N_{S,v} and N_{S,h}, which are connected to operational amplifiers OPᵥ or OPₕ, are connected at their non-inverting input to ground. Negative feedback by means of the resistors R_{S,h} or R_{S,v} is achieved via the output channel Ch_{E} or Ch_{F}.

FIG. 4 shows four sensor chips M₁, M₂, M₃, M₄ connected via Ch_{1A}, Ch_{1B}, Ch_{2A} and Ch_{2B} or Ch_{3A}, Ch_{3B}, Ch_{4A} and Ch_{4,B} as well as the output channels Ch_{3D}, Ch_{3C}, Ch_{1D} and Ch_{1C} or Ch_{4D}, Ch_{4C}, Ch_{2D} and Ch_{2C}.

Analogously, the summing networks N_{S1,v,1}-N_{S2,v,1}, N_{S1,v,2}-N_{S2,v,2}, N_{S1,v,M}-N_{S2,v,M} as well as N_{S3,v,1}-N_{S4,v,1}, N_{S3,v,2}-N_{S4,v,2}, N_{S3,v,M}-N_{S4,v,M} and N_{S3,h,1}-N_{S1,h,1}, N_{S3,h,2}-N_{S1,h,2}, N_{S3,h,N}-N_{S1,h,N} as well as N_{S4,h,1}-N_{S2,h,1}, N_{S4,h,2}-N_{S2,h,2}, N_{S4,h,N}-N_{S2,h,N} are connected via the sensor chips M₁, M₂, M₃, M₄.

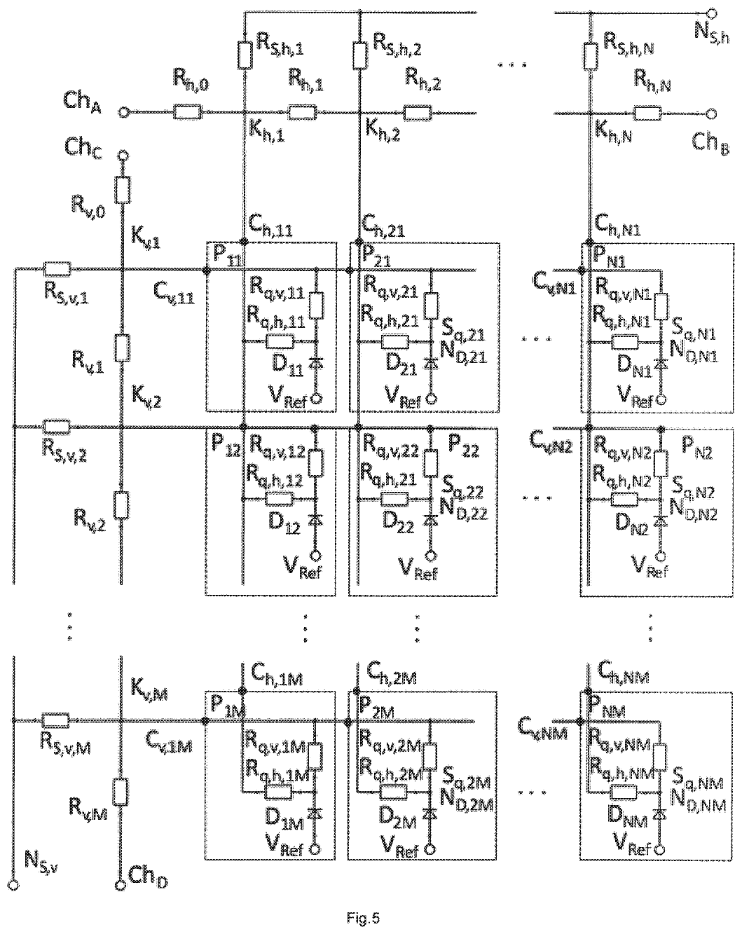

FIG. 5 shows microcells with photodiodes Dₙₘ, which lead into a current divider S_{q,nm}, which is implemented with the quench resistors R_{q,h,nm}, R_{q,v,nm}. The outputs of the current dividers R_{q,h,nm} lead into signal buses N_{S,h,n}, which lead into the nodal points K_{h,n} and, via the series connection R_{h,0}, R_{h,1}, . . . R_{h,N}, into the output channels Ch_{A} and Ch_{B}. The outputs of the current dividers R_{q,v,nm} lead into signal buses N_{S,v,m}, which lead into the nodal points K_{v,m} and, via the series connection R_{v,0}, R_{v,1}, . . . R_{v,M}, into the output channels Ch_{C} and Ch_{D}. The resistors R_{h,0}-R_{h,N} or R_{v,0}-R_{v,M} form a series connection whose ends are the outputs Ch_{A} and Ch_{B} or Ch_{C} and Ch_{D}. The resistors R_{S,h,1}, . . . , R_{S,h,n} and R_{S,v,1}, . . . , R_{S,v,m} are integrated onto the sensor chip, and only the summing networks N_{S,h} and N_{S,v} are led out of the sensor chip.

In FIG. 6, summing networks N_{S,v} and N_{S,h} lead into the operational amplifiers OPᵥ and OPₕ, which are grounded and lead into the output channels Ch_{F} and Ch_{E}. The operational amplifiers are negatively fed back via the resistors R_{S,v} and R_{S,h}.

FIG. 7 shows four sensor chips M₁, M₂, M₃, M₄ via which the output channels Ch_{1A}, Ch_{1B}, Ch_{2A} and Ch_{2B} or Ch_{3A}, Ch_{3B}, Ch_{4A} and Ch_{4,B} and the output channels Ch_{3D}, Ch_{3C}, Ch_{1D} and Chic or Ch_{4D}, Ch_{4C}, Ch_{2D} and Ch_{2C} are connected.

Analogously, the summing networks N_{S1,v}-N_{S4,v} and N_{S1,h}-N_{S4,h} are connected via the sensor chips M₁, M₂, M₃, M₄.

While subject matter of the present disclosure has been illustrated and described in detail in the drawings and foregoing description, such illustration and description are to be considered illustrative or exemplary and not restrictive. Any statement made herein characterizing the invention is also to be considered illustrative or exemplary and not restrictive as the invention is defined by the claims. It will be understood that changes and modifications may be made, by those of ordinary skill in the art, within the scope of the following claims, which may include any combination of features from different embodiments described above.

The terms used in the claims should be construed to have the broadest reasonable interpretation consistent with the foregoing description. For example, the use of the article “a” or “the” in introducing an element should not be interpreted as being exclusive of a plurality of elements. Likewise, the recitation of “or” should be interpreted as being inclusive, such that the recitation of “A or B” is not exclusive of “A and B,” unless it is clear from the context or the foregoing description that only one of A and B is intended. Further, the recitation of “at least one of A, B and C” should be interpreted as one or more of a group of elements consisting of A, B and C, and should not be interpreted as requiring at least one of each of the listed elements A, B and C, regardless of whether A, B and C are related as categories or otherwise. Moreover, the recitation of “A, B and/or C” or “at least one of A, B or C” should be interpreted as including any singular entity from the listed elements, e.g., A, any subset from the listed elements, e.g., A and B, or the entire list of elements A, B and C.

### CITED PRIOR ART

[1]: Gola, A., et al., “A Novel Approach to Position-Sensitive Silicon Photomultipliers: First Results”.
[2]: Schulz, V, et al., “Sensitivity encoded silicon photomultiplier—a new sensor for high-resolution PET-MRI.” Physics in medicine and biology 58.14 (2013): 4733.
[3]: Fischer, P., Piemonte, C., “Interpolating silicon photomultipliers”, NIMPRA, November 2012.
[4]: Espana, S., et al., “DigiPET: sub-millimeter spatial resolution small-animal PET imaging using thin monolithic scintillators”.
[5]: Lerche, Ch. W., et al., “Depth of interaction detection for γ-ray imaging”.
[6]: U.S. Pat. No. 7,476,864 (B2).
[7]: Ito, M., et al., “Positron Emission Tomography (PET) Detectors with Depth-of-Interaction (DOI) Capability”.
[8]: Judenhofer, M. S., et al., “Simultaneous PET-MRI: a new approach for functional and morphological imaging”.
[9]: Ziegler, S. I., et al., “A prototype high-resolution animal positron tomograph with avalanche photodiode arrays and LSO crystals”.
[10]: Balcerzyk, M., et al., “Preliminary performance evaluation of a high resolution small animal PET scanner with monolithic crystals and depth-of-interaction encoding”.
[11]: Balcerzyk, M., et al., “Initial performance evaluation of a high resolution Albira small animal positron emission tomography scanner with monolithic crystals and depth-of-interaction encoding from a user's perspective”.
[12]: Gonzalez Martinez, A. J., et al., “Innovative PET detector concept based on SiPMs and continuous crystals”.
[13]: Siegel, S., et al., “Simple Charge Division Readouts for Imaging Scintillator Arrays using a Multi-Channel PMT”.
[14]: McElroy, D. P., et al., “First Results From MADPET-II: A Novel Detector and Readout System for High Resolution Small Animal PET”.
[15]: Berneking, A., “Characterization of Sensitivity encoded Silicon Photomultiplier for high resolution simultaneous PET/MR Imaging”, Diploma thesis, RWTH Aachen University, Dec. 3, 2012.

1: A sensor chip, comprising:
a plurality of microcells to which an xy position is assigned, composed of a photodiode D_{n,m}, a current divider S_{q,nm}, with outputs S_{q,v,nm}, for the y direction and outputs S_{q,h,nm} for the x direction, the outputs S_{q,h,nm} being equipped with a quenching apparatus R_{q,h,nm} for quenching the current, and the outputs S_{q,v,nm} being equipped with a quenching apparatus R_{q,v,nm} for quenching the current, which divides the generated photocurrent of the diodes D_{n,m} into two equally large fractions,
wherein the microcells are arranged in a sequence of N columns in the x direction x_{n,}=x₁, x₂, x₃, . . . xₙ with n=1, 2, 3, . . . N and M rows in the y direction y_{m,}=y₁, y₂, y₃, . . . yₘ with m=1, 2, 3, . . . M,
wherein the outputs S_{q,h,nm} of the current dividers S_{q,nm} for the x direction are connected to the read-out channels Ch_{A} and Ch_{B} for the x direction, current conductors of the same x position of the sensor chip being connected to the same signal bus N_{S,h,1}, which leads into the read-out channel Ch_{A} and Ch_{B} in the x direction, and
wherein a series connection of x-encoding resistors R_{h,0}, R_{h,1}, R_{h,2}, . . . R_{h,N} is located in the read-out channels Ch_{A} and Ch_{B}, the signal buses N_{S,h,i} leading into nodal points K_{h,n} with n=1, 2, 3, . . . N, which are located between the x-encoding resistors R_{h,0}, R_{h,1}, R_{h,2}, . . . R_{h,N}, thereby effecting linear encoding, the linear encoding being given when the following condition is satisfied:
Q₁(ε)=c₁·ε^{c2}+c₃
Q₂(ε)=c₄·ε^{c3}+c₆
c₁=const.∈(0,∞)
c₄=const.∈(−∞,0)
c₃,c₆=const.∈(−∞,∞)
0.5<c₂,c₅<1.5 (Formula 1)

2: The sensor chip according to claim 1, wherein the outputs of the current dividers S_{q,v,nm} for the y direction are connected to output channels Ch_{C} and Ch_{D} for the y direction, which leads into the read-out channel Ch_{C} and Ch_{D} in the y direction, current conductors of the same y position of the sensor chip being connected to the same signal bus N_{S,v,1}, which leads into the read-out channel Ch_{C} and Ch_{D} in the y direction, and a series connection of y-encoding resistors R_{v,0}, R_{v,1}, R_{v,2}, . . . R_{v,M} is located in the read-out channels Ch_{C} and Ch_{D}, the signal buses N_{S,v,1} leading into nodal points K_{v,m} with m=1, 2, 3, . . . M, which are located between the y-encoding resistors R_{v,0}, R_{v,1}, R_{v,2}, . . . R_{v,M}, thereby effecting linear encoding
Q₁(ε)=c₁·ε^{c2}+c₃
Q₂(ε)=c₄·ε^{c3}+c₆
c₁=const.∈(0,∞)
c₄=const.∈(−∞,0)
c₃,c₆=const.∈(−∞,∞)
0.5<c₂,c₅<1.5 (Formula 1)

3: The sensor chip according to claim 1, wherein multiple photodiodes D_{n,m} are combined with current dividers S_{q,nm} and quenching apparatus R_{q,h,nm} to form a microcell and lead into a signal bus N_{Shn} for the x position.

4: The sensor chip according to claim 1, wherein multiple photodiodes D_{n,m} are combined with current dividers S_{q,nm} and quenching apparatus R_{q,v,nm} to form a microcell and lead into a signal bus N_{Svm} for the y position.

5: The sensor chip according to claim 1, wherein encoding resistance values of the x-encoding resistors R_{h,1}, . . . R_{h,N-1} have the same value.

6: The sensor chip according to claim 1, wherein encoding resistance values for the y-encoding resistors R_{v,1}, . . . R_{v,M-1} have the same value.

7: The sensor chip according to claim 1, wherein the encoding resistors for R_{h,n} and for R_{v,m} have an encoding resistance value between 0.001 ohm and 100 Mohm.

8: The sensor chip according to claim 1, wherein the number N of microcells in the x direction and the number M of microcells in the y direction are different.

9: The sensor chip according to claim 2, wherein encoding resistance values for encoding Ch_{A}, Ch_{B} and Ch_{C}, Ch_{D} are different.

10: The sensor chip according to claim 1, wherein the signal buses N_{S,h,1}, N_{S,h,2} . . . N_{S,h,N} and/or N_{S,v,1}, N_{S,v,2} . . . N_{S,v,M} are fed via summing resistors R_{S,h,n} and/or R_{S,v,m} in summing networks N_{S,h} and/or N_{S,v}, downstream of which an operational amplifier Oₕ, Oᵥ is connected to output channels Ch_{E} and/or Ch_{F}.

11: The sensor chip according to claim 10, wherein the operational amplifiers Oₕ, Oᵥ with the output channels Ch_{E} and/or Ch_{F} are arranged outside the sensor chip.

12: The sensor chip according to claim 10, wherein the summing networks N_{S,h}, N_{S,v} are arranged outside the sensor chip.

13: The sensor chip according to claim 10, wherein the summing resistors R_{S,h,n}, R_{S,v,m} are arranged outside the sensor chip.

14: The sensor chip according to any of claim 1, wherein at least 2 sensor chips in the x direction and/or in the y direction are connected via shared signal buses N_{S,h,1}, N_{S,h,2} . . . N_{S,h,N} and/or N_{S,v,1}, N_{S,v,2} . . . N_{S,v,M}, which lead into summing resistors R_{S,h,n}, R_{S,v,m} which in summing networks N_{S,h}, N_{S,v}.

15: The sensor chip according to claim 14, wherein the resistance values R_{s,h,0} and R_{s,h,N} have the value R_{S,h,n}/2 as well as the resistance values R_{s,v,0} and R_{s,v,M} have the resistance value R_{S,v,m}/2.
